SEQUENCE LISTING (16 sequences)

SEQ ID NO: 1 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 2 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEAALENLVVLNAASVAGAHGILSFLVFFCAAWYIKGRLVPGAAYALYGVWPLLLLLLALPPRAYAMDREMAASCGGAVFVGLILLTLSPHYKLFLARLIWWLQYFITRAEAHLQVWIPPLNVRGGRDAVILLTCAIHPELIFTITKILLAILGPLMVLQAGITKVPYFVRAHGLIRACMLVRKVAGGHYVQMALMKLAALTGTYVYDHLTPLRDWAHAGLRDLAVAVEPVVFSDMETKVITWGADTAACGDIILGLPVSARRGREIHLGPADSLEGQGWRLLAPITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICDECHSTDSTTILGIGTVLDQAETAGARLVVLATATPPGSVTVPHPNIEEVALSSTGEIPFYGKAIPIETIKGGRHLIFCHSKKKCDELAAKLSGLGLNAVAYYRGLDVSVIPTSGDVIVVATDALMTGFTGDFDSVIDCNTCVTQTVDFSLDPTFTIETTTVPQDAVSRSQRRGRTGRGRMGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTTTHPITKYIMACMSADLEVVTSTWVLVGGVLAALAAYCLTTGSVVIVGRIILSGKPAIIPDREVLYREFDEMEECASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEEASEDVVCCSMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 3 - protein, 447 aa, Hepatitis C virus
SGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVC

SEQ ID NO: 4 - protein, 60 aa, Hepatitis C virus
RALPVWARPDYNPPLVETWKKPDYEPPVVHGCPLPPPRSPPVPPPRKKRTVVLTESTLST

SEQ ID NO: 5 - protein, 60 aa, Hepatitis C virus
RAMPIWARPDYNPPLLESWKDPDYVPPVVHGCPLPPAKAPPIPPPRRKRTVVLSESTVSS

SEQ ID NO: 6 - protein, 60 aa, Hepatitis C virus
RALPVWARPDYNPPLVEPWKKPDYEPPVVHGCPLPPPQSPPVPPPRKKRTVVLTESTLPT

SEQ ID NO: 7 - protein, 59 aa, Hepatitis C virus
RALPIWARPDYNPPLVESWKKPDYEPPVVHGCPLPPPRSPPVPPPRKKRTVVLTESTLP

SEQ ID NO: 8 - protein, 13 aa, Hepatitis C virus
WARPDYNPPLVEP

SEQ ID NO: 9 - DNA, 36 bp, Artificial Sequence
  PCR Primer
gtaaaacgacggccagtccgctccatctctcaaggc

SEQ ID NO: 10 - DNA, 37 bp, Artificial Sequence
  PCR Primer
caggaaacagctatgactgcctttggcaagcactgcg

SEQ ID NO: 11 - DNA, 35 bp, Artificial Sequence
  PCR Primer
gtaaaacgacggccagaaccacctgtggtccatgg

SEQ ID NO: 12 - DNA, 37 bp, Artificial Sequence
  PCR Primer
caggaaacagctatgacttacgaccccccttctcrgg

SEQ ID NO: 13 - DNA, 35 bp, Artificial Sequence
  PCR Primer
gtaaaacgacggccagggargaygtcgtgtgctgc

SEQ ID NO: 14 - DNA, 35 bp, Artificial Sequence
  PCR Primer
caggaaacagctatgacattgcctcctccgtacgg

SEQ ID NO: 15 - DNA, 69 bp, Artificial Sequence
  PCR Primer
ttcgctcgagccctgcccgtttgggcgcggccggactacaaccccccgctagtagagccctggaaaaag

SEQ ID NO: 16 - DNA, 25 bp, Artificial Sequence
  PCR Primer
ccatgtatacgacattgagcagcag

(19) United States
(12) Patent Application Publication
(10) Pub. No.: US 2012/0077738 A1
(43) Pub. Date: Mar. 29, 2012

(54) COMPOSITIONS AND METHODS FOR PREDICTING HCV SUSCEPTIBILITY TO ANTIVIRAL AGENTS

(75) Inventors: Robert Striker, Madison, WI (US); Israr Ansari, Middleton, WI (US)

(73) Assignee: Wisconsin Alumni Research Foundation, Madison, WI (US)

(21) Appl. No.: 13/229,271

(22) Filed: Sep. 9, 2011

Related U.S. Application Data
(60) Provisional application No. 61/386,306, filed on Sep. 24, 2010.

(51) Int. Cl. A61K 38/13; A61P 31/14; C40B 40/08; C12Q 1/70; C07H 21/00

(52) U.S. Cl. 514/4.3; 435/5; 536/23.72; 506/17

(57) ABSTRACT

Methods for determining the susceptibility of a hepatitis C virus (HCV) in a patient to anti-viral agents, particularly cyclophilin inhibitors such as cyclosporine A, are disclosed. The methods include determining the amino acid sequence within a region of the HCV NS5A protein and comparing the viral amino acid sequence to that of a reference strain, wherein the existence of at least one variant/mutation in the viral genome is indicative that the virus is more or less susceptible to anti-viral agents. Also disclosed are isolated polynucleotide molecules, replicons, and kits that can be used to assay the susceptibility of hepatitis HCV in a patient to anti-viral agents.

### CROSS-REFERENCE TO RELATED APPLICATIONS

This application claims the benefit of U.S. provisional Application No. 61/386,306, filed on Sep. 24, 2010, which is incorporated by reference herein in its entirety.

### STATEMENT REGARDING FEDERALLY FUNDED RESEARCH OR DEVELOPMENT

Not Applicable.

### FIELD OF THE INVENTION

This invention relates generally to methods and compositions for customizing anti-viral medication treatment regimes for patients infected with hepatitis C virus. In particular, the invention is directed to methods and compositions that facilitate genetic comparisons between certain regions of a given HCV strain and a known consensus sequence to determine the susceptibility of the HCV strain to treatment with certain anti-viral agents.

### BACKGROUND OF THE INVENTION

Hepatitis C virus (HCV) was first characterized in 1989 (Choo et al., 1989, Science 244: 359-362), although its existence had been suspected for many years as the elusive cause of a liver disease referred to as non A-non B hepatitis (“NANBH”), with flu-like symptoms and occurring in many patients years after they receive blood transfusion. HCV is a single-stranded, plus-sense RNA virus of Flaviviridae, which includes viruses that cause bovine diarrhea, hog cholera, yellow fever, and tick-borne encephalitis. The HCV genome is approximately 9.5 kb in size, and is characterized by a unique open reading frame encoding a single poly-protein.

It is estimated that HCV infects about 170 million people worldwide, more than four times those infected with human immunodeficiency virus (“HIV”), and the number of HCV associated deaths may eventually overtake deaths caused by AIDS (Cohen, 1999, Science 285:26-30). The Center for Disease Control (CDC) has calculated that 1.8 percent of the U.S. population may be infected with HCV.

HCV infection is now known to be the leading cause of liver failure in the United States. Approximately 60% of HCV patients develop chronic liver disease and a substantial number of these patients have to undergo liver transplant. Unfortunately, the virus survives in other cells and eventually infects the new liver upon transplant. HCV infected patients have a higher mortality rate than non-HCV infected liver transplant patients at five years, likely due, at least in part, to accelerated HCV infection of the transplanted liver, leading to the recurrence of liver failure.

Immunosuppressive agents, or immunosuppressants, are invariably required for all allografts to blunt the recipient's immune response and minimize rejection. Use of immunosuppressants, however, has been linked to the increase in HCV virulence and in patient morbidity and mortality. This effect is especially pronounced in liver transplantation and is observed to a lesser extent in kidney transplantation.

Contradicting observations, however, have been widely reported with regard to some of the immunosuppressants, especially cyclosporine A (CsA). In some instances, CsA treatments are known to lead to an increase in virulence of HCV in the liver (see e.g. Everson, Impact of immunosuppressive therapy on recurrence of hepatitis C. Liver Transpl, 2002. 8(10 Suppl 1): p. S19-27), yet in other instances, CsA has been shown to inhibit HCV replication in vitro and has been used as a treatment for HCV infection. For example, Nakagawa et al. (Specific inhibition of hepatitis C virus replication by cyclosporin A. Biochem Biophys Res Commun 313(1):42-7. 2004) and Watashi et al. (Cyclosporin A suppresses replication of hepatitis C virus genome in cultured hepatocytes. Hepatology 38(5): 1282-8. 2003) reported that CsA can inhibit HCV replication in vitro through a mechanism apparently unrelated to its immunosuppressive properties. Though CsA does not appear to control HCV effectively in liver transplant recipients, presumably due to its immunosuppressive effects, a study in Japan found that a six-month course of HCV treatment with a combination of CsA and alpha interferon was more effective at achieving sustained virological responses than interferon alone (42/76 [55%] vs. 14/44 [32%]; p=0.01) (Inoue et al., Combined interferon alpha2b and cyclosporin A in the treatment of chronic hepatitis C: controlled trial. J. Gastroenterol 38567-72. 2003). Further research is focused on NIM811, Debio-025, SCY325 and various CsA analogue with varying immunosuppressive activity.

The inconsistency among the various reported research likely involves differences in study design, varying complexity of the patient population, such as differences in how patients respond to immunosuppressants, and other factors. The most likely cause of the inconsistency, however, is the high genetic heterogeneity of the HCV virus. Based upon phylogenetic analysis of the core, EI, and NS5 regions of the viral genome, the HCV virus has been classified into at least six genotypes and more than 30 subtypes dispersed throughout the world (Major and Feinstone, 1997, Hepatology 25: 1527-1538; Clarke, 1997, J. Genl. Virol 78: 2397-2410). It is believed that various genotypes or subtypes of HCV may be susceptible to inhibition by immunosuppressants such as cyclosporine A (CsA), while others may not. However, direct or specific correlation between the genotype of an HCV strain and its susceptibility to immunosuppressant treatment is lacking. As a consequence, currently, modifying CsA treatment of HCV in transplant patients is reactionary, with viral load or increased virulence, as indicated by tissue destruction, being the only indicators of failure of CsA treatment of HCV.

There is, therefore, a need to determine the susceptibility of a viral strain to an anti-viral in a patient, and to anti-viral/immunosuppressive treatment regimens that also prevent graft rejection without leaving the patient vulnerable to excessive morbidity and mortality from HCV infection. There is further a need for tools which physicians can use before and during CsA or other cyclophilin inhibitor treatment to monitor development of anti-viral resistance or susceptibility by the virus, to predict and verify treatment efficacy, and to customize treatment.

### SUMMARY OF THE INVENTION

The present inventors have shown that the antiviral benefit of antiviral agents varies according to variations of the HCV genome and amino acid sequence, and that certain HCV strains display more sensitivity to antiviral agents, including cyclophilin inhibitors such as CsA, than others. Thus, the present invention provides methods and compositions for determining variation and/or mutations in genetic and/or amino acid sequences of HCV to predict the effectiveness of antiviral agents, especially cyclophilin inhibitors, in treating HCV infection in general, and in liver transplant patients in particular.

Accordingly, in one aspect, the invention encompasses a method for determining susceptibility of a hepatitis C virus (HCV) in a sample to an anti-viral agent. The method includes the steps of (1) determining the amino acid sequence within the HCV NS5A region, and (2) comparing the amino acid sequence to that of a reference strain. The existence of at least one mutation/variation in the viral amino acid sequence as compared to the reference strain is indicative that the virus is more or less susceptible to the anti-viral agent.

In one embodiment, the at least one mutation/variation is in a consensus amino acid sequence corresponding to amino acid residues 316-328 of the wild type HCV NS5A region of SEQ ID NO:3. Because length polymorphisms occur in various HCV strains, the amino acid residue numbering of the consensus sequence can vary in different HCV strains. Preferably, the at least one mutation/variation is a proline substitution at the amino acid position corresponding to amino acid residue 328 of SEQ ID NO:3, wherein amino acid residue 328 is typically a threonine or a serine residue in wildtype HCV lineages. Preferably, the mutated/variant consensus sequence is selected from the group consisting of WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, and WARPDYX₄PPX₅X₆X₇X₈, wherein X₁, X₂, X₃, X₄, X₅, X₆, and X₇ can be any amino acid and X₈ is proline, alanine, isoleucine, methionine, or arginine. More preferably, the mutated/variant consensus sequence is WARPDYNPPLVEP.

In certain embodiments, the anti-viral agent is a cyclophilin inhibitor. Non-limiting examples of cyclophilin inhibitors for which the method could be used include Debio-025, SCY-325, and cyclosporine A (CsA). CsA is the preferred cyclophilin inhibitor for which the method is used.

In some embodiments, the sample used in the method is a clinical sample obtained from a HCV infected patient. Preferably, the patient is a liver-transplant patient.

In a second aspect, the invention encompasses an isolated polynucleotide that includes a nucleic acid sequence that encodes for a region within the HCV NS5A protein having at least one mutation/variation in a consensus amino acid sequence corresponding to amino acid residues 316-328 of the reference HCV NS5 region of SEQ ID NO:3, wherein amino acid residue 328 is a threonine or a serine residue in wildtype HCV lineages. Preferably, the mutated/variant consensus sequence encoded by the polynucleotide is WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, or WARPDYX₄PPX₅X₆X₇X₈, wherein X₁, X₂, X₃, X₄, X₅, X₆, and X₇ can be any amino acid and X₈ is proline, alanine, isoleucine, methionine, or arginine. More preferably, the mutated/variant consensus sequence encoded by the polynucleotide is WARPDYNPPLVEP. The invention further encompasses an antiviral agent-susceptible HCV replicon that includes the isolated polynucleotide, and a gene chip including at least two such isolated polynucleotides.

In a third aspect, the invention encompasses a kit including (1) at least one isolated polynucleotide as described above, and (2) a means for determining whether a sample contains a nucleic acid molecule that comprises the nucleotide sequence of the polynucleotide. The means for determining whether a sample contains a nucleic acid molecule that comprises the nucleotide sequence of the polynucleotide may include reagents suitable for a PCR or a hybridization reaction that utilizes the polynucleotide molecule as a primer or a probe.

In a fourth aspect, the invention encompasses a method of monitoring the development of anti-viral agent susceptibility in an HCV patient. The method includes the step of determining the amino acid sequence of a region of the NS5A protein of the HCV polyprotein in a sample from the patient. The appearance of a mutation/variant as described previously is indicative that the HCV has developed increased or decreased susceptibility to the anti-viral agent. Preferably, the patient is a liver transplant patient afflicted by HCV infection.

In a fifth aspect, the invention encompasses a method for managing HCV treatment in a liver-transplant patient. The method includes the steps of (1) determining whether the HCV in the patient is susceptible to a given anti-viral agent, as described previously, and (2) administering to the patient a suitable anti-viral agent or combination of agents accordingly.

In a sixth aspect, the invention encompasses a method for screening for anti-viral pharmaceutical compounds. The method includes the steps of (1) applying a candidate compound to a cell culture that includes an antiviral agent-susceptible replicon as described previously, and (2) determining whether the candidate compound inhibits viral replication or viral protein synthesis. A candidate that shows inhibitory effects is an anti-viral compound.

Other objects, features and advantages of the present invention will become apparent after review of the specification, claims, and data and figures set forth herein.

### BRIEF DESCRIPTION OF THE DRAWINGS

FIG. 1 depicts results for 63 HIV/HCV genotype 1 transplanted patients.

FIG. 2 illustrates results for 63 HIV/HCV genotype 1 transplanted patients (indicating patients with one third genome population sequenced pre- and post-transplant).

FIG. 3 depicts results for 63 HIV/HCV genotype 1 transplanted patients (indicating specific patient identifiers).

FIG. 4 depicts viremia associated with patient 203-002.

FIG. 5 illustrates selection of 4 in vivo derived mutations conferring relative CsA resistance in vitro.

FIG. 6 provides a schematic of the HCV replicon and methodology utilized by the present inventors and described in the Examples section.

FIG. 7 depicts transient replication of HCV replicon in Huh 7 cells, which is suppressed by CsA. CsA is less able to suppress the post CsA exposure chimeric.

FIG. 8 shows that individual mutants do not alter the fitness of the replication. Pretransplant chimeric is susceptible to CsA (green triangle). A single amino acid change from serine 328 to proline 328 significantly effects CsA treatment (orange square).

FIG. 9 shows replication efficiency of replicon with proline and pre-proline to serine changes.

FIG. 10 illustrates HCV NS5A C-terminal region binding to cyclophilin.

FIG. 11 illustrates CsA susceptibility of HCV 1b chimeric replicon along with gene sequences derived from pre-transplant case. The solid line (no CsA treatment) vs dashed lines (CsA treated) indicate replicons at a particular time. The pre-transplant sample containing Pro at position 328 is more susceptible to CsA relative to the pre-transplant sample containing Thr at position 328.

FIG. 12 illustrates CsA susceptibility of naturally occurring variants in the context of an HCV 1b replicon containing NS5A C-terminal domain derived from 1a genotype.

FIG. 13 provides a comparison of CsA susceptibility of Pro and Ser at amino acid residue position 328 of SEQ ID NO:3 with NS5A C-terminal gene sequences derived from pre- and post-transplant cases exposed to CsA.

FIG. 14 provides a comparison of CsA susceptibility of Pro at amino acid residue position 328 of SEQ ID NO:3 along with NS5A C-terminal gene sequences derived from pre- and post-transplant cases exposed to CsA.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising”, “including”, and “having” can be used interchangeably. The term “polypeptide” and the term “protein” are used interchangeably herein.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

Unless otherwise indicated, the art-accepted standard single letter amino acid codes are used herein to identify specific amino acids and the amino acid substitutions of the present invention. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “nucleic acid” typically refers to large polynucleotides. A “polynucleotide” means a single strand or parallel and anti-parallel strands of a nucleic acid. Thus, a polynucleotide may be either a single-stranded or a double-stranded nucleic acid. A polynucleotide is not defined by length and thus includes very large nucleic acids, as well as short ones, such as an oligonucleotide The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. An “isolated” polynucleotide or polypeptide is one that is substantially pure of the materials with which it is associated in its native environment. By substantially free, is meant at least 50%, at least 55%, at least 60%, at least 65%, at advantageously at least 70%, at least 75%, more advantageously at least 80%, at least 85%, even more advantageously at least 90%, at least 91%, at least 92%, at least 93%, at least 94%, at least 95%, at least 96%, at least 97%, most advantageously at least 98%, at least 99%, at least 99.5%, at least 99.9% free of these materials.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”. Sequences on a DNA strand which are located 5′ to a reference point on the DNA are referred to as “upstream sequences”. Sequences on a DNA strand which are 3′ to a reference point on the DNA are referred to as “downstream sequences.”

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase.

“Probe” refers to a polynucleotide that is capable of specifically hybridizing to a designated sequence of another polynucleotide. “Probe” as used herein encompasses oligonucleotide probes. A probe may or may not provide a point of initiation for synthesis of a complementary polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., detectable moieties, such as chromogenic, radioactive or fluorescent moieties, and used as detectable agents.

### II. The Invention

The inventors have discovered that variation or mutation of the amino acid sequence in a region of the NS5A protein of the HCV genome renders an increase or decrease in the susceptibility of HCV to anti-viral cyclophilin inhibitors, including CsA. To precisely define the specific mutation/variation that increases susceptibility to CsA, a reference amino acid sequence for wild type HCV 1a (NCBI accession no. AF009606.1, SEQ ID NO:1) and wild type HCV 1b (NCBI accession no. AJ238799.1, SEQ ID NO:2) are provided herein. A standard reference NS5A amino acid sequence is a 447 amino acid region that includes amino acid residues 1973-2419 of SEQ ID NO:1 (SEQ ID NO:3). The mutation that indicates increased susceptibility of HCV to anti-viral agents, particularly to cyclophilin inhibitors such as CsA, is a single proline, alanine, isoleucine or methionine substitution at amino acid residue 2300 of SEQ ID NO:1, which corresponds to amino acid residue 328 of SEQ ID NO:3. The mutation that indicates decreased susceptibility of HCV to anti-viral agents, particularly to cyclophilin inhibitors such as CsA, is a single arginine substitution at amino acid residue 2300 of SEQ ID NO:1, which corresponds to amino acid residue 328 of SEQ ID NO:3. A serine substitution at amino acid residue 2300 of SEQ ID NO:1, which corresponds to amino acid residue 328 of SEQ ID NO:3, is common and does not influence susceptibility of HCV to anti-viral agents.

The substituted amino acid residue is the thirteenth residue of a thirteen amino acid consensus sequence that the skilled artisan would recognize as being analogous across varying HCV amino acid sequences. The consensus sequence corresponds to amino acid residues 318-328 of SEQ ID NO:3, and amino acid residues 2288-2300 of SEQ ID NO:1 and SEQ ID NO:2. As HCV is subject to frequent mutation, there can be significant variation among individual HCV sequences. The consensus sequence is represented as WAX₁PX₂X₃X₄PPX₅X₆X₇X₈ where WAX₁PX₂X₃X₄ typically is WARPDYN, but can vary in one of the four amino acids labeled X₂-X₄. In addition, X₅, X₆, and X₇ can individually vary. In the mutation referred to above, X₈ is proline, alanine, isoleucine, or methionine, signaling that the strain is more cyclophilin inhibitor sensitive than strains having other amino acids at the X₈ position. In the mutation referred to above, X₈ is arginine, signaling that the strain is less cyclophilin inhibitor sensitive than strains having other amino acids at the X₈ position. Amino acid residue 328 is typically a threonine or serine residue in wildtype HCV lineages.

In the Examples below, the inventors report the variations in the amino acid sequences in this region for a patient (patient 203-002) having low viremia immediately post-transplant, but greatly increased viremia after approximately sixty weeks. The following four sequences are offered for comparison. Each of the sequences begins at amino acid residue 311 of the NS5A region (corresponding to amino acid residue 311 of SEQ ID NO:3 or amino acid residue 2283 of SEQ ID NO:1 or SEQ ID NO:2).

The following is this portion of the sequence for HCV 1a and HCV 1b. The consensus sequence is underlined. Note that position 328 (the last underlined residue) is threonine or serine, respectively.

HCV 1a:
(SEQ ID NO: 4)
RALPVWARPDYNPPLVETWKKPDYEPPVVHGCPLPPPRSPPVPPPRKKRTVVLTESTLST
HCV 1b:
(SEQ ID NO: 5)
RAMPIWARPDYNPPLLESWKDPDYVPPVVHGCPLPPAKAPPIPPPRRKRTVVLSESTVSS

The pre-transplant sequence of patient 203-002, who exhibited low viremia for about a year, is shown below. Note that position 328 (the last underlined residue) is substituted with proline. Without being bound by theory, the inventors have associated this substitution with increased susceptibility to anti-viral agents, and have confirmed that this variant/mutant is particularly sensitive to CsA when tested in a tissue culture HCV replicon model. In contrast, the HCV in patient 203-002 acquired a post-transplant mutation that correlated with a steep rise in viremia. The post-transplant sequence is also shown below. Note that position 328, the last underlined residue (but there can be length polymorphisms), has reverted to the wild type serine residue. Accordingly, the HCV has reduced susceptibility to anti-viral agents such as CsA.

Pre-transplant:
(SEQ ID NO: 6)
RALPVWARPDYNPPLVEPWKKPDYEPPVVHGCPLPPPQSPPVPPPRKKRTVVLTESTLPT
Post-transplant:
(SEQ ID NO: 7)
RALPIWARPDYNPPLVESWKKPDYEPPVVHGCPLPPPRSPPVPPPRKKRTVVLTESTLP

Substitution in either direction from proline to a non-proline (associated with resistance) or from a non-proline to a proline (associated with susceptibility) is possible.

Accordingly, in a first aspect, the invention encompasses a method for determining susceptibility of a hepatitis C virus (HCV) in a sample to an anti-viral agent, the method comprising determining the amino acid sequence within the HCV NS5A region and comparing said amino acid sequence to that of a wild-type strain, wherein the existence of at least one mutation in the viral amino acid sequence is indicative that the virus is more or less susceptible to the anti-viral agent. Preferably, the at least one mutation is in a consensus amino acid sequence corresponding to amino acid residues 316-328 of the wild type HCV NS5A region of SEQ ID NO:3; more preferably, the at least one mutation is a proline, alanine, isoleucine, methionine, or arginine substitution at the amino acid corresponding to residue 328 of SEQ ID NO:3.

The mutated consensus sequence is selected from the group consisting of WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, WARPDYX₄PPX₅X₆X₇X₈, X₁, X₂, X₃, X₄, X₅, X₆, and X₇ can be any amino acid and X₈ is proline, alanine, isoleucine, methionine, or arginine. In a preferred embodiment, the mutated consensus sequence is WARPDYNPPLVEP (SEQ ID NO:8).

In certain embodiments, the anti-viral agent is a cyclophilin inhibitor. Non-limiting examples cyclophilin inhibitors include Debio-025, SCY-325, and cyclosporine A (CsA). Preferably, the cyclophilin inhibitor is CsA.

Preferably, the sample is a clinical sample obtained from a HCV infected patient, including without limitation a liver-transplant patient. Clinical samples useful in the practice of the methods of the invention can be any biological sample from which any of genomic DNA, mRNA, unprocessed RNA transcripts of genomic DNA or combinations of the three can be isolated. As used herein, “unprocessed RNA” refers to RNA transcripts which have not been spliced and therefore contain at least one intron. Suitable biological samples are removed from human patient and include, but are not limited to, blood, buccal swabs, hair, bone, and tissue samples, such as skin or biopsy samples. Biological samples also include cell cultures established from an individual.

Genomic DNA, mRNA, and/or unprocessed RNA transcripts are isolated from the biological sample by conventional means known to the skilled artisan. See, for instance, Sambrook et al. (2001, Molecular Cloning: A Laboratory Manual, Cold Spring Harbor Laboratory Press, Cold Spring Harbor, N.Y.) and Ausubel et al. (eds., 1997, Current Protocols in Molecular Biology, John Wiley & Sons, New York). The isolated genomic DNA, mRNA, and/or unprocessed RNA transcripts is used, with or without amplification, to detect a mutation relevant to the invention.

A variety of methodologies may be adapted by routine optimization to facilitate polypeptide or nucleotide sequence determination of HCV NS5A regions of interest. For example, nucleotide sequence information may be obtained by direct DNA sequencing of HCV NS5A region nucleic acid contained in a biological sample obtained from a patient of interest (e.g., a blood sample). The assay may be adapted to use a variety of automated sequencing procedures (Naeve et al., 1995, Biotechniques 19:448-453), including sequencing by mass spectrometry (see, e.g., PCT International Publication No. WO 94/16101; Cohen et al., 1996, Adv. Chromatogr. 36:127-162; and Griffin et al., 1993, Appl. Biochem. Biotechnol. 38:147-159). Traditional sequencing methods may also be used, such as dideoxy-mediated chain termination method (Sanger et al., 1975, J. Molec. Biol. 94: 441; Prober et al. 1987, Science 238: 336-340) and the chemical degradation method (Maxam et al., 1977, PNAS 74: 560).

A preferred sequencing method for detection of a single nucleotide change is pyrosequencing. See, for instance, Ahmadian et al., 2000, Anal. Biochem, 280:103-110; Alderborn et al., 2000, Genome Res. 10:1249-1258 and Fakhrai-Rad et al., 2002, Hum. Mutat. 19:479-485. Pyrosequencing involves a cascade of four enzymatic reactions that permit the indirect luciferase-based detection of the pyrophosphate released when DNA polymerase incorporates a dNTP into a template-directed growing oligonucleotide. Each dNTP is added individually and sequentially to the same reaction mixture, and subjected to the four enzymatic reactions. Light is emitted only when a dNTP is incorporated, thus signaling which dNTP in incorporated. Unincorporated dNTPs are degraded by apyrase prior to the addition of the next dNTP. The method can detect heterozygous individuals in addition to heterozygotes. Pyrosequencing uses single stranded template, typically generated by PCR amplification of the target sequence. One of the two amplification primers is biotinylated thereby enabling streptavidin capture of the amplified duplex target. Streptavidin-coated beads are useful for this step. The captured duplex is denatured by alkaline treatment, thereby releasing the non-biotinylated strand.

In a second aspect, the invention encompasses an isolated polynucleotide comprising a nucleic acid sequence that encodes for a region within the HCV NS5A protein having at least one mutation in a consensus amino acid sequence corresponding to amino acid residues 316-328 of the wild type HCV NS5 region of SEQ ID NO:3, wherein the mutated consensus sequence encoded by the polynucleotide is selected from the group consisting of WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, WARPDYX₄PPX₅X₆X₇X₈, and wherein X₁, X₂, X₃, X₄, X₅, X₆, and X₇ can be any amino acid and X₈ is proline, alanine, isoleucine, methionine, or arginine. In some such embodiments, the mutated consensus sequence encoded by the polynucleotide is WARPDYNPPLVEP (SEQ ID NO:8).

Two or more such polynucleotides may be included in a diagnostic kit, microarray, or gene chip used to carry out detection methods according to the invention. The polynucleotide may also be incorporated in an antiviral agent-susceptible HCV replicon.

Amplification of a polynucleotide sequence according to the invention may be carried out by any method known to the skilled artisan. See, for instance, Kwoh et al., (1990, Am. Biotechnol. Lab. 8, 14-25) and Hagen-Mann, et al., (1995, Exp. Clin. Endocrinol. Diabetes 103:150-155). Amplification methods include, but are not limited to, polymerase chain reaction (“PCR”) including RT-PCR, strand displacement amplification (Walker et al., 1992, PNAS 89, 392-396; Walker et al., 1992, Nucleic Acids Res. 20, 1691-1696), strand displacement amplification using Phi29 DNA polymerase (U.S. Pat. No. 5,001,050), transcription-based amplification (Kwoh et al., 1989, PNAS 86, 1173-1177), self-sustained sequence replication (“35R”) (Guatelli et al., 1990, PNAS 87, 1874-1878; Mueller et al., 1997, Histochem. Cell Biol. 108:431-437), the Q.beta. replicase system (Lizardi et al., 1988, BioTechnology 6, 1197-1202; Cahill et al., 1991, Clin., Chem. 37:1482-1485), nucleic acid sequence-based amplification (“NASBA”) (Lewis, 1992, Genetic Engineering News 12 (9), 1), the repair chain reaction (“RCR”) (Lewis, 1992, supra), and boomerang DNA amplification (or “BDA”) (Lewis, 1992, supra).

PCR may be carried out in accordance with known techniques. See, e.g., Bartlett et al., eds., 2003, PCR Protocols Second Edition, Humana Press, Totowa, N.J. and U.S. Pat. Nos. 4,683,195; 4,683,202; 4,800,159; and 4,965,188. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained. The amplified target polynucleotide may be used in one of the detection assays described elsewhere herein to identify the mutation present in the amplified target polynucleotide sequence.

In a third aspect, the invention encompasses a kit comprising at least one isolated polynucleotide as described above and a means for determining whether a sample contains a nucleic acid molecule that comprises the nucleotide sequence of the polynucleotide. The means for determining whether a sample contains a nucleic acid molecule may include reagents suitable for a PCR or a hybridization reaction that utilizes the polynucleotide molecule as a primer or a probe.

More specifically, the kit may contain at least one pair of amplication primers that is used to amplify a target HCV NS5A nucleotide region containing one of the mutations identified in the invention. The amplification primers are designed based on the sequences provided herein for the upstream and downstream sequence flanking the mutation. In a preferred embodiment, the amplification primers will generate an amplified double-stranded target polynucleotide between about 50 base pairs to about 600 base pairs in length and, more preferably, between about 100 base pairs to about 300 base pairs in length. In another preferred embodiment, the mutation is located approximately in the middle of the amplified double-stranded target polynucleotide.

The kit may further contain a detection probe designed to hybridize to a sequence 3′ to the mutation on either strand of the amplified double-stranded target polynucleotide. In one variation, the detection probe hybridizes to the sequence immediately 3′ to the mutation on either strand of the amplified double-stranded target polynucleotide but does not include the mutation. This kit variation may be used to identify the mutation by pyrosequencing or a primer extension assay. For use in pyrosequencing, one of the amplification primers in the kit may be biotinylated and the detection probe is designed to hybridize to the biotinylated strand of the amplified double-stranded target polynucleotide. For use in a primer extension assay, the kit may optionally also contain fluorescently labeled ddNTPs. Typically, each ddNTP has a unique fluorescent label so they are readily distinguished from each other.

Any of the above kit variations may optionally contain one or more nucleic acids that serve as a positive control for the amplification primers and/or the probes. Any kit may optionally contain an instruction material for performing risk diagnosis.

In a fourth aspect, the invention encompasses a method of monitoring the development of anti-viral agent susceptibility in an HCV patient, the method comprising determining the amino acid sequence of a region of the NS5A protein of the HCV polyprotein in a sample from the patient, wherein the appearance of the mutation/variant characterized previously is indicative that the HCV has developed increased or decreased susceptibility to the anti-viral agent. Again, the method could be used with a liver transplant patient afflicted by HCV infection. Such a method could be extended to the management of HCV treatment in a liver-transplant patient. Such a method would include the steps of determining whether the HCV in the patient is susceptible to a given anti-viral agent, and administering to the patient a suitable anti-viral agent or combination of agents accordingly.

Detection of proline or alanine at position X₈ in the consensus sequence WAX₁PX₂X₃X₄PPX₅X₆X₇ X₈ (i.e., amino acid residue 328 of SEQ ID NO:3, and amino acid residue 2300 of SEQ ID NO:1 and SEQ ID NO:2) of HCV in a patient sample indicates that a cyclophilin inhibitor should be included in the antiviral regimen provided to that patient. In contrast, detection of arginine at position X₈ in the consensus sequence of HCV in a patient sample indicates that a cyclophilin inhibitor should not be included in the antiviral regimen provided to that patient.

In a fifth aspect, the invention encompasses a method for screening for anti-viral pharmaceutical compounds. The method includes the steps of applying a candidate compound to a cell culture that comprises an antiviral agent-susceptible replicon as described previously, and determining whether the candidate compound inhibits viral replication or viral protein synthesis. A candidate that shows inhibitory effects is a demonstrated anti-viral compound.

The embodiments described here and in the following example are for illustrative purposes only, and various modifications or changes apparent to those skilled in the art are included within the scope of the invention. The terminology used to describe particular embodiments is not intended to limit the scope of the present invention, which is limited only by the claims. The following examples are offered to illustrate, but not to limit, the scope of the present invention.

### Example

### Proline Variation Correlates with HCV Susceptibility to Cyclophilin Inhibition Post Transplant

Introduction:

HCV is one of the most common indications for liver transplant worldwide. After liver transplantation though HCV, reinfection is nearly universal, and the disease is typically more aggressive in the now immunosuppressed patient than it was pretransplant. The optimal immunosuppression regimen for HCV infected transplant patients are unclear. Since HCV replication requires the host cofactor cyclophilin, the cyclophilin inhibitor and immunosuppressant CsA has been suggested as preferred over the more common immunosuppressant tacrolimus. Some prospective studies but not all have failed to detect a benefit from CsA. Strains of HCV may not be are equally susceptible to cyclophilin inhibition, and the selection of CsA resistant HCV post transplant could also obscure a benefit. By examining a cohort of HIV/HCV infected transplant patients with an atypically high use of CsA we show here a critical variant of HCV NS5A is correlated with CsA susceptibility in cell culture and in patients.

Materials and Methods:

Cells, media and chemicals. The Huh 7.5 cells were propagated in Advanced DMEM (Invitrogen, cat. 12491023) containing 1× Glutamine (Invitrogen, cat. 25030164), 1× Penicillin/Streptomycin (Invitrogen, cat. 15140122) and 1× non-essential amino acids (Invitrogen, cat. 11140050). Earlier we received the Huh7.5 cells and the Con1 HCV replicon from Dr. Charles M. Rice, the Rockefeller University, NY[1]. For our studies, the neomycin resistance gene in this replicon was replaced with a renilla luciferase-neo fusion gene which was amplified from another HCV replicon provided by Dr. N. Kato [2] and termed Con1-Luciferase-Neomycin (Con1LN) replicon which was used in our lab before [3,4]. CsA was purchased from Sigma-Aldrich (St. Louis, cat. C3662) and resuspended in absolute ethanol before use.

Serum samples and sequencing. The serum/plasma samples were subjected to RNA isolation using RNeasy kit (Qiagen cat. 52904). Using standard HCV based primers tagged with M13-forward and M13-reverse sequences, RT-PCR was performed. The PCR products were subjected to sequencing using M13-forward and M13-reverse primers and only consensus sequences were taken in account. The following primers were used for generating PCR products and subsequent sequencing.

F8_for
(SEQ ID NO: 9)
GTAAAACGACGGCCAGTCCGCTCCATCTCTCAAGGC
F8_rev
(SEQ ID NO: 10)
CAGGAAACAGCTATGACTGCCTTTGGCAAGCACTGCG
F9_for
(SEQ ID NO: 11)
GTAAAACGACGGCCAGAACCACCTGTGGTCCATGG
F9_rev
(SEQ ID NO: 12)
CAGGAAACAGCTATGACTTACGACCCCCCTTCTCRGG
F10_for
(SEQ ID NO: 13)
GTAAAACGACGGCCAGGGARGAYGTCGTGTGCTGC
F10_rev
(SEQ ID NO: 14)
CAGGAAACAGCTATGACATTGCCTCCTCCGTACGG

Primers used to create mutation. The patient derived HCV genome was PCR amplified using the primers listed below. The expected size PCR product was digested with XhoI and BstZ17I restriction sites and cloned directionally in Con1b-LN (previously described from our lab) replicon. The mutant replicons were tested for CsA sensitivity as described.

Forward primer:
(SEQ ID NO: 15)
5′ TTCGCTCGAGCCCTGCCCGTTTGGGCGCGGCCGGACTACAACCCC
CCGCTAGTAGAGCCCTGGAAAAAG 3′
Reverse primer:
(SEQ ID NO: 16)
5' CCATGTATACGACATTGAGCAGCAG 3′

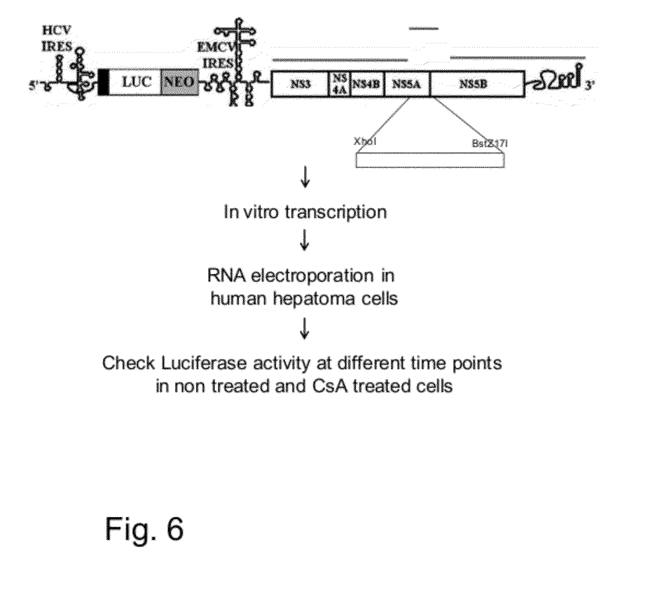

Genetic manipulation of HCV replicon. The Con1LN replicon was digested with XhoI and BstZ17I restriction enzymes (New England Biolabs) and corresponding fragment from HCV genotype 1a spanning from amino acid 2282 to 2420 (amino acid position with reference to H77 HCV genome AF009606) was cloned into the replicon, termed Con1LN-chimera. This Chimeric replicon was tested for its replication efficiency and found replication competent in tissue culture system. The Chimeric replicon was further utilized for cloning homologous fragments derived from pre- and post-liver transplant individuals infected with HCV. FIG. 6 illustrates the replicon and general methodology utilized in this study.

RNA transcription and transient replication Assay. Replicon DNA was linearized with XbaI (New England Biolabs) and transcribed using a MEGAscript T7 kit (Applied Biosystems cat. AMB1334) as per manufacturer's protocol. Six microgram of purified RNA was electroporated into 2×10⁶ Huh7.5 cells using Gene Pulser) (cell electroporation system 250V, 850 uF, ∞R, 4 cm cuvette (Bio-Rad, CA). The electroporated cells were divided into two halves and seeded into twenty-four well plates. After the cells were attached the media was aspirated and replaced with fresh media for the first half, while the other half was treated with 0.5 ug/ml of CsA. The cells were further incubated and harvested from both sets at five different time points (24, 48, 72, 96 and 120 hrs) and renilla luciferase activity was monitored as per manufacturer's protocol. In brief, the cells were lysed with 100 μl of Renilla Lysis buffer supplied with the Renilla Luciferase kit (Promega, WI, cat. E2810). 5 μl of clarified cell lysate was mixed with 45 μl of Renilla Luciferase Assay buffer and read in triplicate on a Glomax 20/20 Luminometer (Promega, WI, USA). The average of three independent assays was calculated and data was analyzed.

Results:

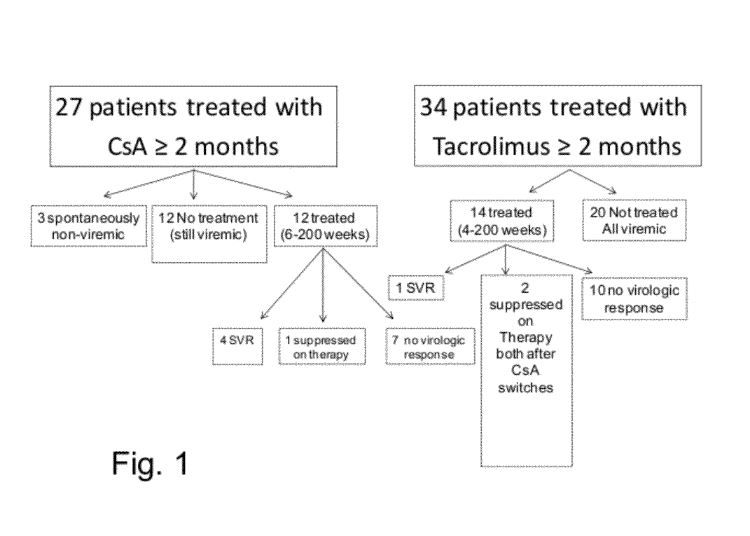

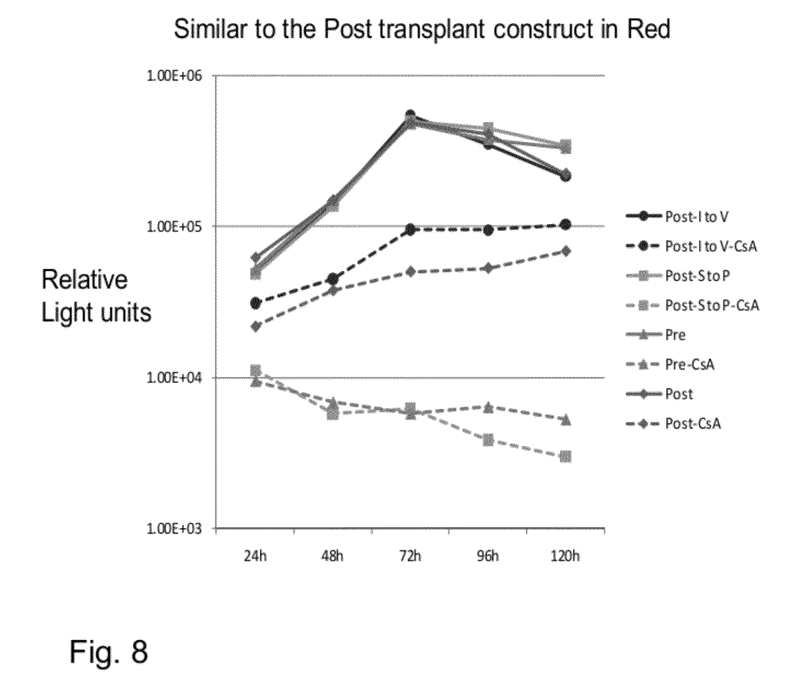

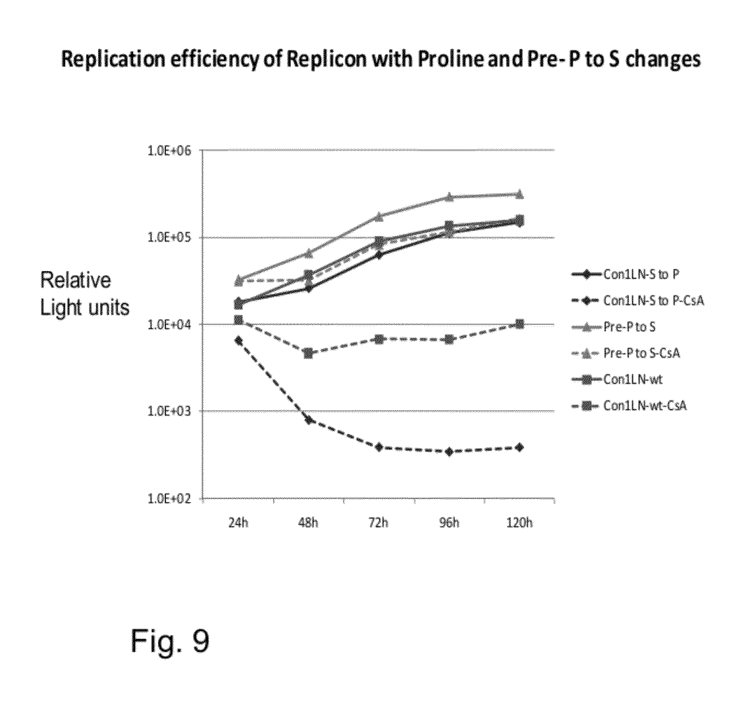

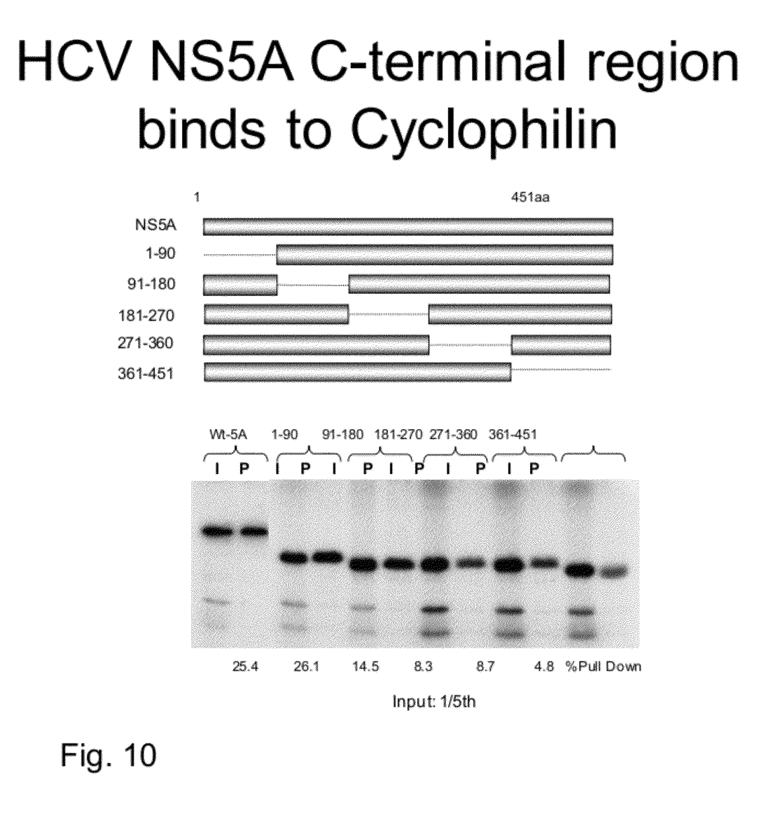

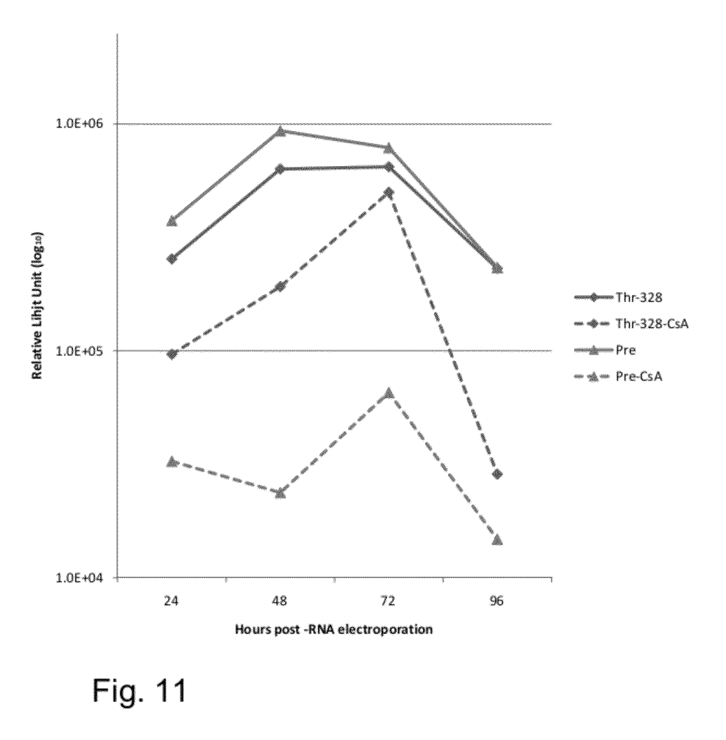

Calcineurin Inhibition Use posttransplant in HIV/HCV infected patients. Eighty HIV/HCV coinfected patients received liver transplants as part of the HIV and transplantation trial from 2003-2009. Of these patients 61 were infected with genotype 1 HCV and received only a liver (not liver+kidney) and are further described here and in the corresponding data shown in FIGS. 1-14. Both calcineurin inhibitor use and HCV treatment post transplant were per transplant center's discretion although centers were encouraged to use CsA due to the dependence of both HIV and HCV on Cyclophilin A.

Referring to FIG. 1, thirty-four patients were treated immediately post transplant with Tacrolimus, and of these, fourteen received some interferon based anti-HCV therapy (median duration 24 weeks, average duration 48 weeks). Only one patient out of these 14 treated patients achieved a negative HCV PCR 6 months or more after HCV therapy was stopped (Sustained Virologic Response, SVR). Two patients treated initially with Tacrolimus eventually achieved a nondetectable HCV PCR after being switched from Tac to CsA. See Appendix A.

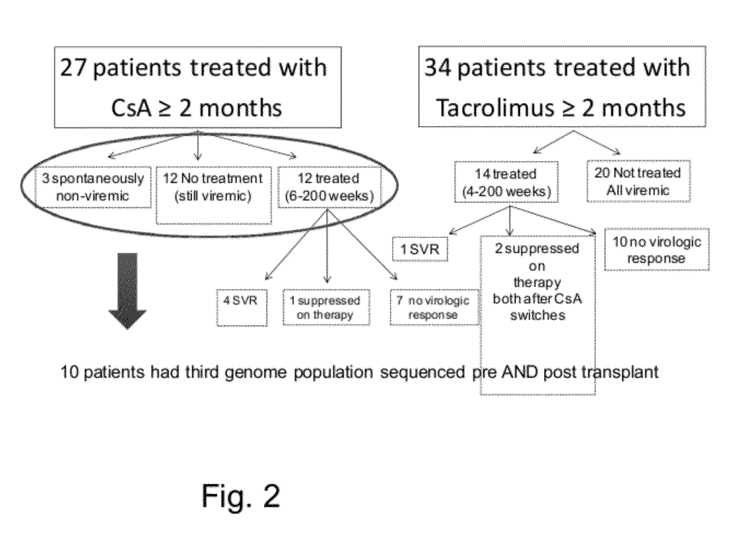

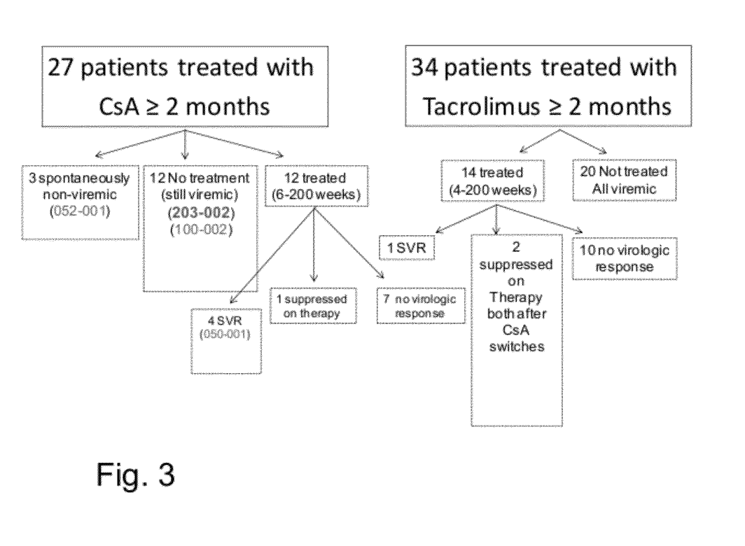

Twenty-seven patients were treated with CsA immediately post transplant. Three of these patients became persistently non-viremic without therapy (052-001, 052-115, 055-005; see FIGS. 2-3). Twelve of these CsA treated patients received some interferon based anti-HCV therapy, although two of these twelve received therapy after the calcineurin inhibitor was switched from CsA to Tacrolimus. Two of the eight taking CsA while on interferon (500-001, 052-127) achieved a nondetectable HCV PCR test off therapy and one is currently non-viremic on therapy (052-180). Three other CsA treated patients were found to be non-viremic post transplant without any HCV therapy, at least two of which was viremic post transplant (055-005, 052-155, 052-001). Seven patients treated with CsA immediately post transplant failed interferon therapy failed although two of these had also switched to Tacrolimus, while 13 patients started on Tacrolimus failed interferon therapy. See FIG. 3.

Sequence Analysis of CsA treated HIV/HCV infected patients. We were interested whether patients that were viremic on CsA therapy developed specific mutations due to the inhibition of Cyclophilin activity. HCV was amplified from pretransplant banked serum as well as postransplant. The earliest posttransplant samples were 12 weeks and the latest were 104 weeks post transplant. Samples from nine patients treated with CsA could be amplified both pre and post transplant. All paired samples were subjected to both dN/dS analysis and phylogenetic analysis. For all nine patients the sequence pre and post transplant were more closely related to each other than to any of the other sequences.

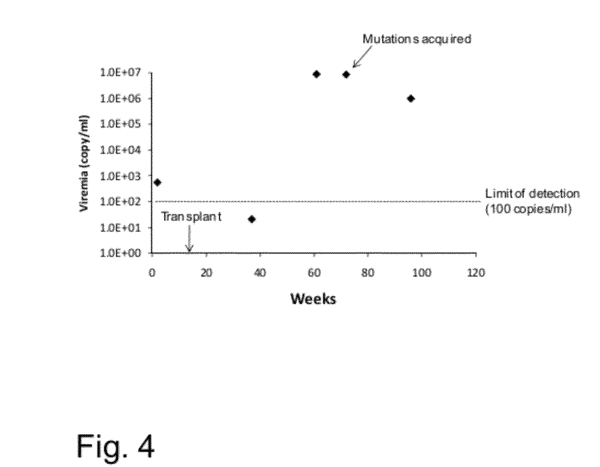

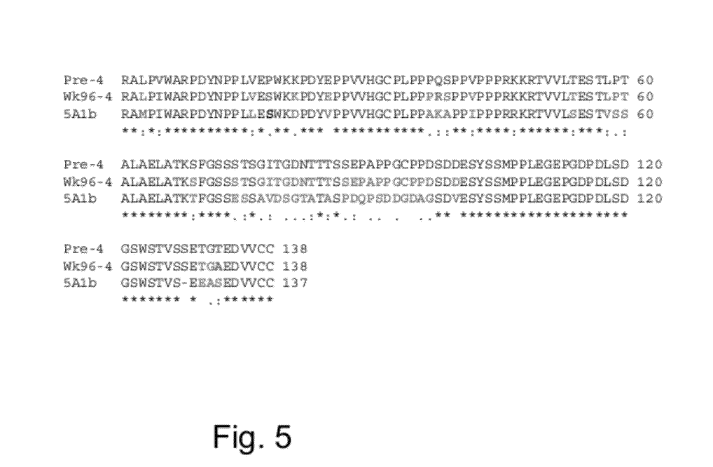

While no two sequence pairs revealed underwent similar evolution, four sequence pairs did show unusual variation in domains 2 and 3 of NS5A, as cell culture replicon experiments had predicted. See FIG. 4-5. Eight of these 9 patients were genotype 1a. The genotype 1b patients acquired mutations in NS5A at residues 320 but was not clearly associated with CsA resistance by replicon analysis, while the selection of a proline to serine change at position 328 in patient 203-002 was associated with less replicon susceptibility to CsA.

Discussion

While HAART has had a dramatic effect on the quality of life and mortality of HIV infected patients, for HIV+patients with ESLD life expectancy remains short (Miro 2007 J of HIV Therapy) even in the post HAART era. The outcomes for HIV+patients for kidney transplant and for non-HCV related liver transplant are comparable to those in the non-HIV+population, but ˜90% of the HIV+ patients that have received liver transplants have HCV and the main cause of death in these patients is HCV reoccurrence (Miro). At the same time the mortality of HIV/HCV patients with ESLD who do not receive liver transplants or are on the waiting list is high (40% survival at 2 years (Pineda 2005). The optimal strategy appears to be to either cure HCV prior to liver transplantation (if it remains necessary) or shortly thereafter (clearly difficult to do with the currently available anti-HCV therapy). Whether immunosuppressive regimens post transplant should be different in either HIV+ patients or HCV+ patients much less coinfected patients has been the subject of tremendous debate and remains unsettled.

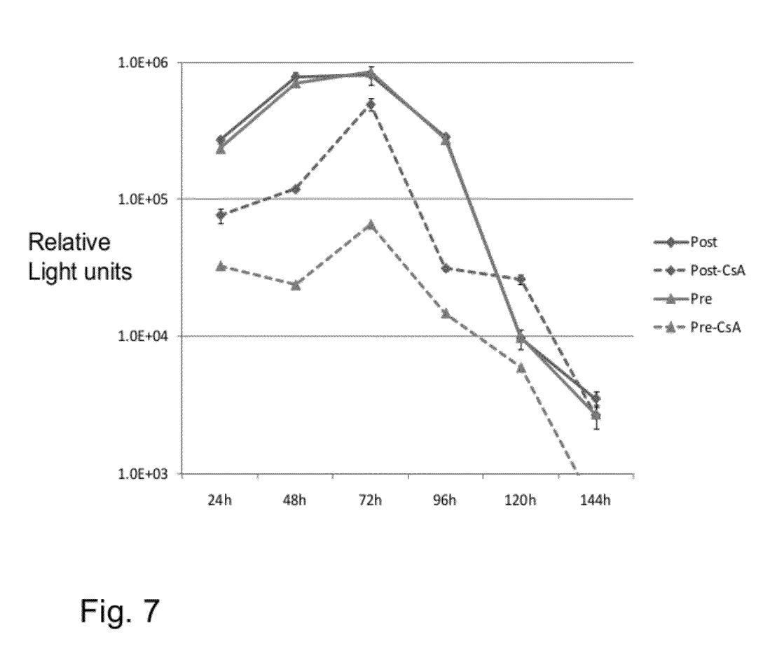

The Con1LN-1a chimeric replicon was manipulated to contain alanine, isoleucine, methionine, or arginine residues at amino acid position 328 relative to SEQ ID NO:3. Alanine, isoleucine, methionine, and arginine are naturally present at amino acid position 328 relative to SEQ ID NO:3 within the HCV genotype 1a but these residues are present at very low frequencies relative to threonine or serine residues at the same amino acid position. CsA susceptibility of replicons containing alanine, isoleucine, methionine, or arginine residues at amino acid position 328 of SEQ ID NO:3 was tested as described above and in FIGS. 7-14. CsA susceptibility of the 1b-1a chimeric replicon was from maximum to minimum in the order of: proline>alanine>serine>methionine>isoleucine>arginine. These findings further confirm that a proline residue at amino acid position 328 results in increased susceptibility to anti-viral CsA treatment.

While the proline substitution at position 328 relative to SEQ ID NO: 3 made a 1b-1a chimeric replicon more CsA susceptible, it also increased susceptibility of the 1b con1 replicon from its baseline. Approximately 10% patients infected with genotype 1b and 5% of genotype 1a strains have position 328 as a proline preexisting as the most common variant as patient 203-002. This variant likely explains why this patient did not have detectable viremia until 60 weeks post transplant. It is expected that this variant will also correlate with higher susceptibility to more potent cyclophilin inhibitors such as Debio-025 and SCY325.

Additional Variation at Position 328 Correlates with HCV Susceptibility to Cyclophilin Inhibition.

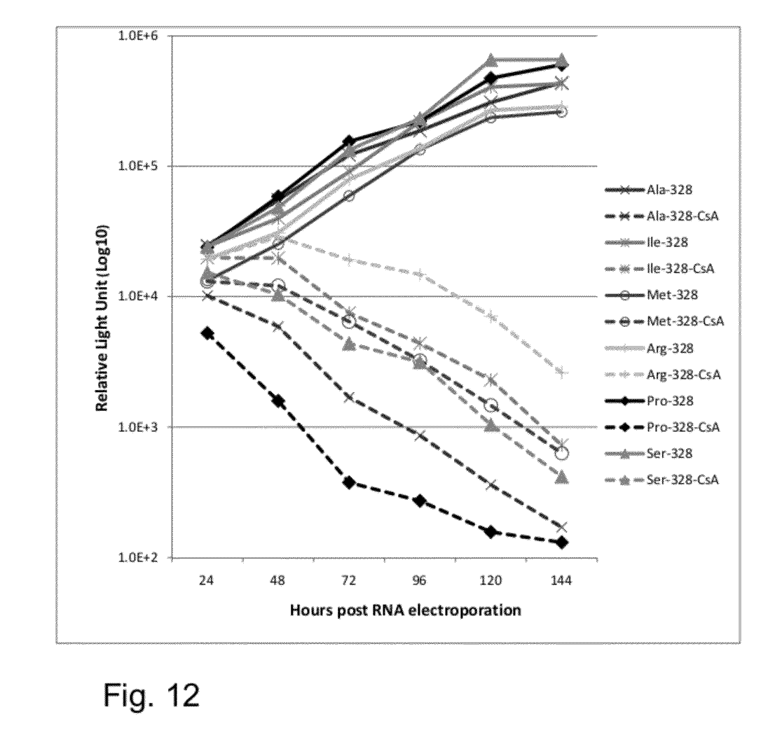

Referring now to FIG. 12, the chimeric replicon was mutated from Thr to Ala, Ile, Met, Arg, Pro, and transient replication assay was performed as described before. Note the solid line (no CsA treated) versus dashed lines (CsA treated) replicons at a particular time. We noticed Pro is suppressed much more than the Arg or Ile indicating that this amino acid position's involvement in cyclophilin susceptibility in HCV genotype, 1a and 1b.

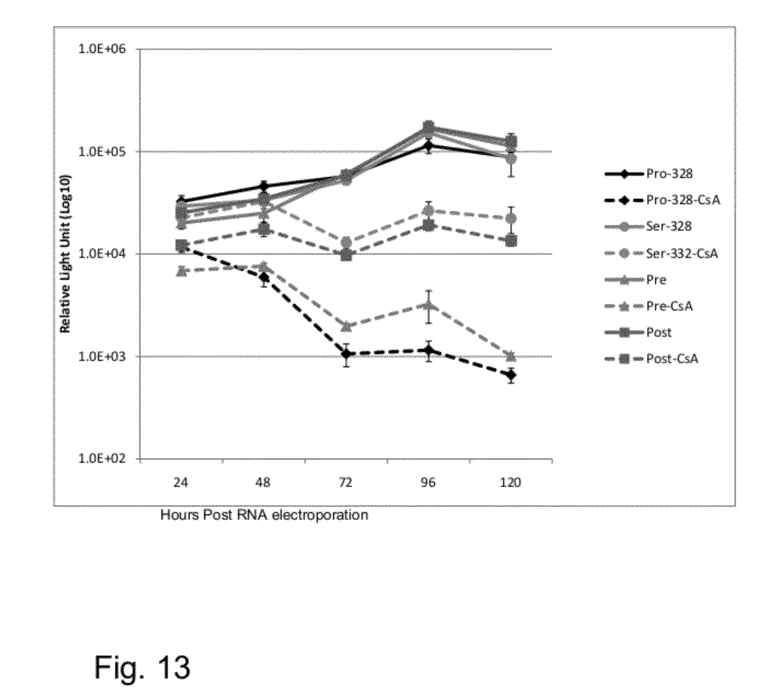

Referring to FIG. 13, the PCR fragments spanning C-terminal domain of NS5A were amplified from pre- and post-transplant/CsA treated patient who acquired 4 mutations in that region. The fragments were cloned into an HCV replicon and replication capacity was monitored as described before. The HCV replicon was also engineered to contain Pro and Ser at 328 amino acid position and replication efficiency was compared. We observed the replicon carrying Pro at amino acid 328 amino along with the replicon carrying gene sequences derived from pre-transplant patient were most sensitive to CsA treatment. The above date further confirms the involvement of this amino acid position in cyclophilin susceptibility.

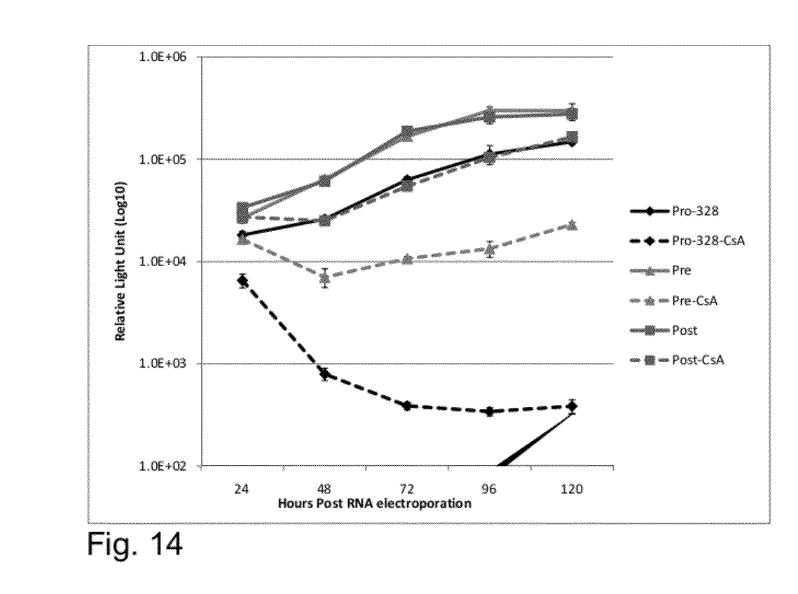

As shown in FIG. 14, the HCV 1b replicon was engineered to contain Pro at 328 amino acid position and replication efficiency was compared along with pre- and post-transplant. As expected, the replicons carrying Pro at amino acid 328 along with the replicon carrying gene sequences derived from pre-transplant patient were most sensitive to CsA treatment.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms thereof as come within the scope of the following claims.

### REFERENCES

1. Blight K J, Kolykhalov A A, Rice C M (2000) Efficient initiation of HCV RNA replication in cell culture. Science 290: 1972-1974.
2. Ikeda M, Abe K, Dansako H, Nakamura T, Naka K, et al. (2005) Efficient replication of a full-length hepatitis C virus genome, strain O, in cell culture, and development of a luciferase reporter system. Biochem Biophys Res Commun 329: 1350-1359.
3. Fernandes F, Ansari I U, Striker R. cyclosporine inhibits a direct interaction between cyclophilins and hepatitis C NS5A. PLoS One 5: e9815.
4. Fernandes F, Poole D S, Hoover S, Middleton R, Andrei A C, et al. (2007) Sensitivity of hepatitis C virus to cyclosporine A depends on nonstructural proteins NS5A and NS5B. Hepatology 46: 1026-1033.

We claim:

1. A method for determining susceptibility of a hepatitis C virus (HCV) in a sample to an anti-viral agent, the method comprising determining the amino acid sequence within the HCV NS5A region and comparing said amino acid sequence to that of a reference strain, wherein the existence of at least one mutation/variation in the viral amino acid sequence is indicative that the virus is more or less susceptible to the anti-viral agent.

2. The method of claim 1, wherein the at least one mutation/variation is in a consensus amino acid sequence corresponding to amino acid residues 316-328 of the wild type HCV NS5A region of SEQ ID NO:3.

3. The method of claim 2, wherein the at least one mutation/variation is a proline, alanine, isoleucine, methionine or arginine substitution at the amino acid corresponding to amino acid residue 328 of SEQ ID NO:3.

4. The method of claim 3, wherein the mutated/variant consensus sequence is selected from the group consisting of WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, and WARPDYX₄PPX₅X₆X₇X₈, and wherein X₁, X₂, X₃, X₄, X₅, X₆, and X₇ can be any amino acid and X₈ is proline, alanine, isoleucine, methionine, or arginine.

5. The method of claim 4, wherein the mutated/variant consensus sequence is WARPDYNPPLVEP.

6. The method of claim 1, wherein the anti-viral agent is a cyclophilin inhibitor.

7. The method of claim 6, wherein the cyclophilin inhibitor is selected from the group consisting of Debio-025, SCY-325, and cyclosporine A (CsA).

8. The method of claim 7, wherein the cyclophilin inhibitor is CsA.

9. The method of claim 1, wherein the sample is a clinical sample obtained from a HCV infected patient.

10. The method of claim 9, wherein the patient is a liver-transplant patient.

11. An isolated polynucleotide comprising a nucleic acid sequence that encodes for a region within the HCV NS5A protein having at least one mutation/variation in a consensus amino acid sequence corresponding to amino acid residues 316-328 of the reference HCV NS5 region of SEQ ID NO:3, wherein the mutated/variant consensus sequence encoded by the polynucleotide is selected from the group consisting of WARPDYNPPX₅X₆X₇X₈, WAX₁PDYNPPX₅X₆X₇X₈, WARPX₂YNPPX₅X₆X₇X₈, WARPDX₃NPPX₅X₆X₇X₈, and WARPDYX₄PPX₅X₆X₇X₈, and wherein X₁, X₂, X₃, X₄, X₅, X₆, and X₇ can be any amino acid and X₈ is proline, alanine, isoleucine, methionine or arginine.

12. The isolated polyneucleotide of claim 11, wherein the mutated/variant consensus sequence encoded by the polynucleotide is WARPDYNPPLVEP.

13. A gene chip comprising at least two isolated polynucleotides according to claim 11.

14. A kit comprising at least one isolated polynucleotide of claim 11, and a means for determining whether a sample contains a nucleic acid molecule that comprises the nucleotide sequence of the polynucleotide.

15. The kit according to claim 14, wherein the means comprises reagents suitable for a PCR or a hybridization reaction that utilizes the polynucleotide molecule as a primer or a probe.

16. A method of monitoring the development anti-viral agent susceptibility in an HCV patient, the method comprising determining the amino acid sequence of a region of the NS5A protein of the HCV polyprotein in a sample from the patient, wherein the appearance of the mutation/variant described in claim 2 is indicative that the HCV has developed increased or decreased susceptibility to the anti-viral agent.

17. The method according to claim 16, wherein the patient is a liver transplant patient afflicted by HCV infection.

18. A method for managing HCV treatment in a liver-transplant patient, the method comprising determining whether the HCV in the patient is susceptible to a given anti-viral agent, and administering to the patient a suitable anti-viral agent or combination of agents accordingly.

19. An antiviral agent-susceptible HCV replicon, comprising the isolated polynucleotide of claim 11.

20. A method for screening for anti-viral pharmaceutical compounds, the method comprising applying a candidate compound to a cell culture that comprises an antiviral agent-susceptible replicon according to claim 19, and determining whether the candidate compound inhibits viral replication or viral protein synthesis, wherein a candidate that shows inhibitory effects is an anti-viral compound.
